(19) United States
(12) Patent Application Publication
(10) Pub. No.: US 2010/0286518 A1
(43) Pub. Date: Nov. 11, 2010

(54) ULTRASOUND SYSTEM AND METHOD TO DELIVER THERAPY BASED ON USER DEFINED TREATMENT SPACES

(75) Inventors: Warren Lee, Niskayuna, NY (US); Dhiraj Arora, Niskayuna, NY (US); Cynthia Elizabeth Landberg Davis, Niskaynuna, NY (US); Ying Fan, Niskayuna, NY (US); Chistopher Robert Hazard, Niskayuna, NY (US); Lowell Scott Smith, Niskayuna, NY (US); Kai Erik Thomenius, Clifton Park, NY (US)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY, SCHENECTADY, NY (US)

(21) Appl. No.: 12/463,783

(22) Filed: May 11, 2009

(51) Int. Cl. A61B 8/00; A61N 7/00

(52) U.S. Cl. 600/439

(57) ABSTRACT

An ultrasound imaging and therapy system is provided that includes an ultrasound probe and a diagnostic module to control the probe to obtain diagnostic ultrasound signals from a region of interest (ROI) of the patient. The ROI includes adipose tissue and the diagnostic module generates a diagnostic image of the ROI based on the ultrasound signals obtained. The system also includes a display to display the image of the ROI and a user interface to accept user inputs to designate a treatment space within the ROI that corresponds to the adipose tissue. The display displays the treatment space on the image. The system also includes a therapy module to control the probe to deliver, during a therapy session, a therapy to a treatment location based on a therapy parameter. The treatment location is within the treatment space defined by the user inputs.

### CROSS-REFERENCE TO RELATED APPLICATIONS

This application includes subject matter that is similar to the subject matter described in U.S. patent Application having Attorney Docket No. 235615 (555-0004US), entitled “ULTRASOUND SYSTEM AND METHOD TO AUTOMATICALLY IDENTIFY AND TREAT ADIPOSE TISSUE.” and Attorney Docket No. 235610 (555-0005US), entitled “ULTRASOUND SYSTEM AND METHOD TO DETERMINE MECHANICAL PROPERTIES OF A TARGET REGION,” both of which are filed contemporaneously herewith and are incorporated by reference in their entirety.

### BACKGROUND OF THE INVENTION

The subject matter herein relates generally to diagnostic imaging and therapy systems that provide diagnostic imaging and treatment of a region of interest in a patient, and more particularly, to ultrasound systems that image and treat adipose tissue.

Various body contouring systems exist today that attempt to remove or destroy fatty tissue (or adipose tissue) from a person's body. Some systems may be invasive, such as liposuction, where a device is inserted into the body and physically removes adipose tissue through suction. Other systems may be non-invasive. For example, in one non-invasive system high-intensity focused ultrasound (HIFU) signals are directed toward a region within the adipose tissue. The HIFU signals may at least partially liquefy the adipose tissue through lysing or causing cavitation or thermal damage of the cells within the adipose tissue.

However, since the ultrasound signals may have a harmful effect on the non-adipose tissue, it is important for a user of a HIFU system to know and control where treatment has been provided within the body of a patient. In one known system, a user draws an outline of a region on a surface of the body where treatment will be provided and also applies markers to the surface around or within the outline on the body of the patient. A video camera is positioned over the body and oriented to view the surface of the patient's skin where therapy is applied. The HIFU system tracks the progress of the therapy based upon the location of the outline on the body and the markers.

The HIFU system described above has certain limitations. For example, the HIFU system may only display the surface of the patient's skin and does not provide a visual representation or image of the volume of the body under the surface. Consequently, the above HIFU system does not provide control for localizing therapy to certain regions under the surface of the skin. Further, the above conventional HIFU system also does not know or determine where non-adipose tissue may be located with respect to the adipose tissue. The HIFU system may also not confirm that therapy has been delivered to the desired regions.

Accordingly, there is a need for ultrasound imaging and therapy systems that indicate where, within a volume of the patient, therapy has been provided or will be provided. Furthermore, there is a need for systems that facilitate a user of the system in identifying a treatment space beneath the surface and applying treatment to the space.

### BRIEF DESCRIPTION OF THE INVENTION

In one embodiment, an ultrasound imaging and therapy system is provided that includes an ultrasound probe and a diagnostic module to control the probe to obtain diagnostic ultrasound signals from a region of interest (ROI) of the patient. The ROI includes adipose tissue and the diagnostic module generates a diagnostic image of the ROI based on the ultrasound signals obtained. The system also includes a display to display the image of the ROI and a user interface to accept user inputs to designate a treatment space within the ROI that corresponds to the adipose tissue. The display displays the treatment space on the image. The system also includes a therapy module to control the probe to deliver, during a therapy session, a therapy to a treatment location based on a therapy parameter. The treatment location is within the treatment space defined by the user inputs.

In another embodiment, a method for delivering therapy to a region of interest (ROI) in a patient is provided. The method includes obtaining diagnostic ultrasound signals from the ROI. The ROI includes adipose tissue. The diagnostic module generates a diagnostic image of the ROI based on the ultrasound signals obtained. The method also includes accepting user inputs to designate a treatment space within the ROI that corresponds to the adipose tissue. The method further includes displaying the image and the treatment space on the image on a display. Also, the method includes providing therapy to a treatment location based on a therapy parameter. The treatment location is within the treatment space defined by the user inputs.

### BRIEF DESCRIPTION OF THE DRAWINGS

FIG. 1 is a block diagram of an ultrasound system formed in accordance with an embodiment of the invention.

FIG. 2 is a block diagram of a diagnostic module in the ultrasound system of FIG. 1 formed in accordance with an embodiment of the invention.

FIG. 3 is a block diagram of a therapy module in the ultrasound system of FIG. 1 formed in accordance with an embodiment of the invention.

FIG. 4 illustrates a window presented on a display of FIG. 1 that displays a treatment space of a region of interest.

FIG. 5 shows the window in FIG. 3 as the ultrasound system delivers therapy to the treatment space.

FIG. 6 is an image of a C-plane view of the region of interest.

FIG. 7 illustrates an ultrasound system in accordance with one embodiment that includes a tracking system and a registering system.

FIG. 8 illustrates transducer arrays that may be used with a probe in accordance with various embodiments.

FIG. 9 illustrates an ultrasound system in accordance with one embodiment that includes a device for removing adipose tissue from a patient during a therapy session.

FIG. 10 is a flowchart illustrating a method in accordance with one embodiment.

FIG. 11 illustrates a hand carried or pocket-sized ultrasound imaging system that may be configured to display a region of interest during a therapy session in accordance with various embodiments.

FIG. 12 illustrates a console-based ultrasound imaging system provided on a movable base that may be configured to display a region of interest during a therapy session in accordance with various embodiments.

FIG. 13 is a block diagram of exemplary manners in which embodiments of the invention may be stored, distributed, and installed on computer readable medium.

### DETAILED DESCRIPTION OF THE INVENTION

Exemplary embodiments that are described in detail below include ultrasound systems and methods for imaging and treating a region of interest (ROI). The ROI may include adipose tissue and/or non-adipose tissue, such as muscle tissue, bone, tissue of organs, and blood vessels. The system may display the ROI so that an operator or user of the system can distinguish the adipose tissue and the non-adipose tissue and/or the system may automatically differentiate the adipose tissue and the non-adipose tissue prior to treating. Treatment of the ROI may include providing high-intensity focused ultrasound (HIFU) signals to treatment locations within the ROI. For example, HIFU signals may be directed to treatment locations within the adipose tissue to at least partially liquefy the adipose tissue. Liquefication may occur through cell lysis, cavitation, and/or thermal damage in the adipose tissue.

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

It should be noted that although the various embodiments may be described in connection with an ultrasound system, the methods and systems described herein are not limited to ultrasound imaging. In particular, the various embodiments may be implemented in connection with different types of medical imaging, including, for example, magnetic resonance imaging (MRI) and computed-tomography (CT) imaging. Further, the various embodiments may be implemented in other non-medical imaging systems, for example, non-destructive testing systems, such as airport screening systems.

A technical effect of the various embodiments of the systems and methods described herein include generating an image of a ROI and accepting user inputs to designate a treatment space within the ROI that corresponds to adipose tissue. Another technical effect may include providing therapy to treatment locations and automatically moving the treatment location between multiple points (or treatment sites) within the treatment. In some embodiments, another technical effect includes analyzing the diagnostic ultrasound signals and automatically differentiating adipose tissue from non-adipose tissue. Other technical effects may be provided by the embodiments described herein.

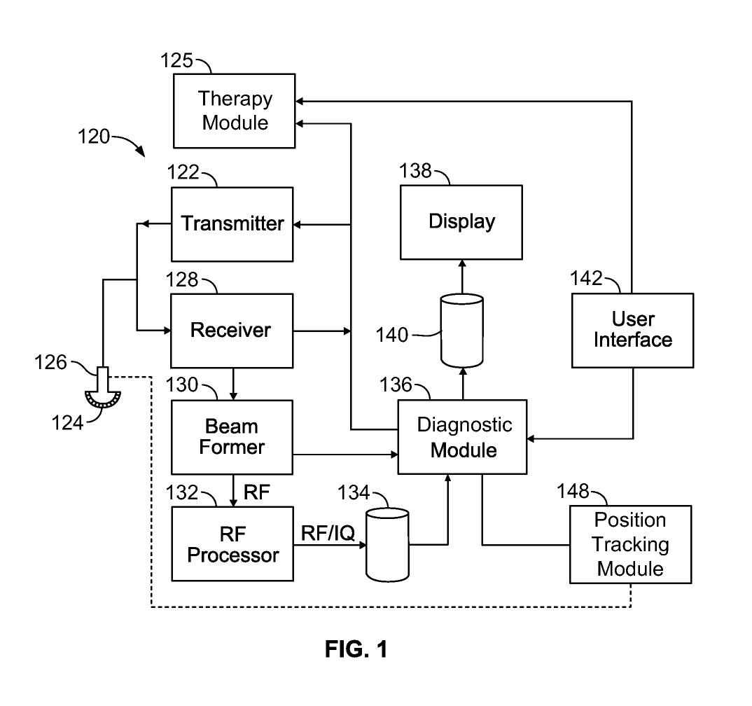

FIG. 1 is a block diagram of an exemplary ultrasound imaging and therapy system 120 in which the various embodiments can display and provide therapy to a ROI as described in more detail below. The ultrasound system 120 includes a transmitter 122 that drives an array of transducer elements 124 (e.g., piezoelectric crystals) within a probe 126 to emit pulsed ultrasonic signals into a body or volume. The pulsed ultrasonic signals may be for imaging and for therapy of the ROI. For example, the probe 126 may deliver low energy pulses during imaging and high energy pulses during therapy. A variety of geometries may be used and the probe 126 may be provided as part of, for example, different types of ultrasound probes.

The imaging signals are back-scattered from structures in the body, for example, adipose tissue, muscular tissue, blood cells, veins or objects within the body (e.g., a catheter or needle) to produce echoes that return to the elements 124. The echoes are received by a receiver 128. The received echoes are provided to a beamformer 130 that performs beamforming and outputs an RF signal. The RF signal is then provided to an RF processor 132 that processes the RF signal. Alternatively, the RF processor 132 may include a complex demodulator (not shown) that demodulates the RF signal to form IQ data pairs representative of the echo signals. The RF or IQ signal data may then be provided directly to a memory 134 for storage (e.g., temporary storage). Optionally, the output of the beamformer 130 may be passed directly to the diagnostic module 136.

The ultrasound system 120 also includes a processor or diagnostic module 136 to process the acquired ultrasound information (e.g., RF signal data or IQ data pairs) and prepare frames of ultrasound information for display on a display 138. The diagnostic module 136 is adapted to perform one or more processing operations according to a plurality of selectable ultrasound modalities on the acquired ultrasound information. Acquired ultrasound information may be processed in real-time during a scanning or therapy session as the echo signals are received. Additionally or alternatively, the ultrasound information may be stored temporarily in the memory 134 during a scanning session and processed in less than real-time in a live or off-line operation. An image memory 140 is included for storing processed frames of acquired ultrasound information that are not scheduled to be displayed immediately. The image memory 140 may comprise any known data storage medium, for example, a permanent storage medium, removable storage medium, etc.

The diagnostic module 136 is connected to a user interface 142 that controls operation of the diagnostic module 136 as explained below in more detail and is configured to receive inputs from a user. The display 138 includes one or more monitors that present patient information, including diagnostic and therapeutic ultrasound images to the user for review, diagnosis, analysis, and treatment. The display 138 may automatically display, for example, a 2D, 3D, or 4D ultrasound data set stored in the memory 134 or 140 or currently being acquired, which data set is also displayed with a graphical representation (e.g., an outline of a treatment space or a marker within the treatment space). One or both of the memory 134 and the memory 140 may store 3D data sets of the ultrasound data, where such 3D data sets are accessed to present 2D and 3D images. For example, a 3D ultrasound data set may be mapped into the corresponding memory 134 or 140, as well as one or more reference planes. The processing of the data, including the data sets, may be based in part on user inputs, for example, user selections received at the user interface 142.

The diagnostic module 136 is configured to receive user imaging commands for outlining or otherwise providing an overlay that indicates a treatment space within the ROI. The diagnostic module 136 may also receive user therapy commands (e.g., through the user interface 142) regarding how to apply therapy to treatment locations within the ROI. The therapy commands may include therapy parameters and the like. The diagnostic module 136 communicates with a therapy module 125 that is configured to control the probe 126 during a therapy session. The diagnostic module 136 is configured to control the probe 126 to obtain diagnostic ultrasound signals from the ROI, and the therapy module 125 is configured to deliver a therapy to the treatment locations based on one or more therapy parameters. The therapy module 125 may automatically move the treatment location between multiple points based on user inputs.

The delivery of therapy may be based upon a therapy parameter. A therapy parameter includes any factor or value that may be determined by the system 120 or any input that may be entered by the user that affects the therapy applied to the ROI. For example, a therapy parameter may include a transducer parameter that relates to the configuration or operation of the transducer elements 124 or probe 126. Examples of a transducer parameter include a focal region depth, a focal region size, an ablation time for each point within the ROI that receives therapy, an energy level of the therapy signals, and a rate of focal region movement within the ROI during the therapy session. The transducer parameters may also include a frequency or intensity of the therapy ultrasound signals, power, peak rarefactional pressure, pulse repetition frequency and length, duty cycle, depth of field, wave form used, speed of beam movement, density of beam, cavitation priming pulse, and general pulse sequence parameters. Also, therapy parameters may include anatomical parameters, such as the location, shape, thickness, and orientation of adipose tissue and non-adipose tissues. An anatomical parameter may also include a density of the adipose tissue and the non-adipose tissues. Furthermore, therapy parameters include the type of probe 126 used during the therapy session. The age, gender, weight, ethnicity, genetics, or medical history of the patient may also be therapy parameters. After therapy has been applied to the treatment space, the system 120 or the operator may adjust the therapy parameters before applying therapy to the treatment space again or another treatment space.

In operation, the system 120 acquires data, for example, volumetric data sets by various techniques (e.g., 3D scanning, real-time 3D imaging, volume scanning, 2D scanning with transducers having positioning sensors, freehand scanning using a voxel correlation technique, scanning using 2D or matrix array transducers, etc.). The data may be acquired by moving the probe 126, such as along a linear or arcuate path, while scanning the ROI. At each linear or arcuate position, the probe 126 obtains scan planes that are stored in the memory 134. The probe 126 also may be mechanically moveable within the ultrasound transducer.

Optionally, the system 120 may include a position tracking module 148 that tracks a position of the probe 126 and communicates the position to the diagnostic module 136. A position of the probe 126 may be tracked relative to a reference point on or near the patient, a marker, and the like. As will be described in greater detail below, the position of the probe 126 may be used to indicate, to the user, regions of the patient that have already been treated, are being treated, or have yet to be treated.

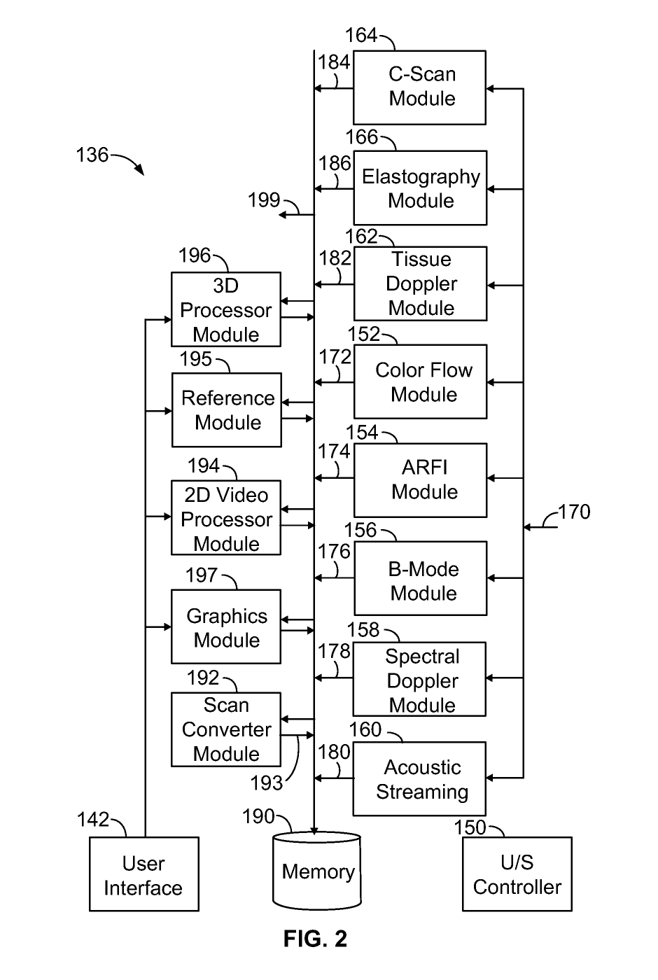

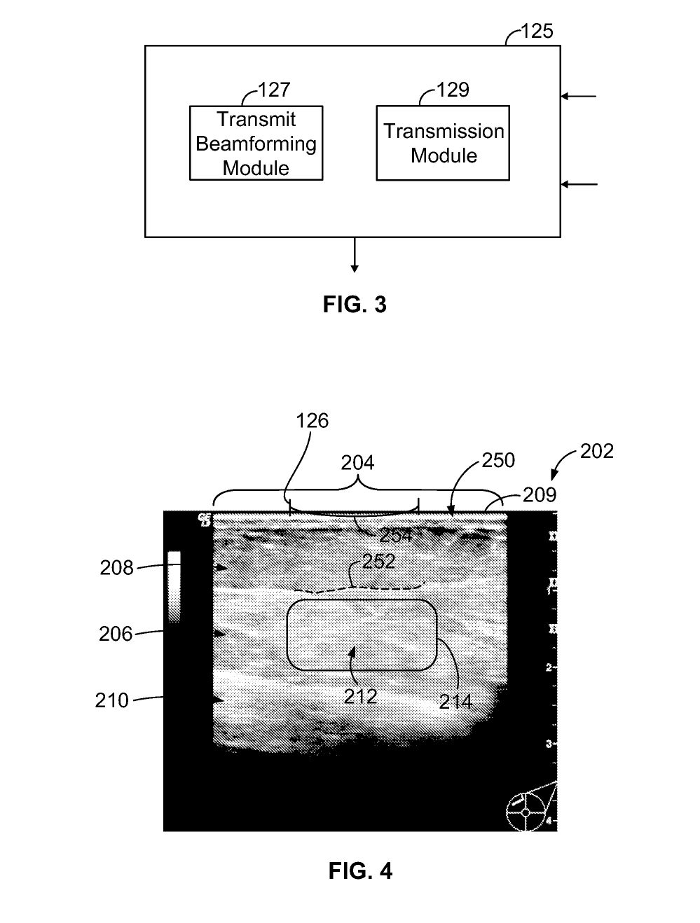

FIG. 2 is an exemplary block diagram of the diagnostic module 136 of FIG. 1, and FIG. 3 is an exemplary block diagram of the therapy module 125. The therapy module 125 may be coupled to the diagnostic module 136 and the user interface 142. The therapy module 125 and the diagnostic module 136 may also be a common module or processor. The therapy module 125 includes a steering or transmit beamforming module 127 and a transmission module 129. The steering module 127 is configured to control the location and movement of a focal spot or region generated by the transducer elements 124. For example, the steering module 127 may control electronic or mechanical steering of the probe to move the focal region of a therapy beam within the treatment space or between different treatment spaces. The transmission module 129 is configured to drive the transducer elements 124 (or only a portion or subset of the transducer elements 124) in delivering energy pulses to the ROI for imaging and therapy.

The therapy and diagnostic modules 125 and 136 are illustrated conceptually as a collection of modules, but may be implemented utilizing any combination of dedicated hardware boards, DSPs, processors, etc. Alternatively, the modules of FIGS. 2 and 3 may be implemented utilizing an off-the-shelf PC with a single processor or multiple processors, with the functional operations distributed between the processors. As a further option, the modules of FIGS. 2 and 3 may be implemented utilizing a hybrid configuration in which certain modular functions are performed utilizing dedicated hardware, while the remaining modular functions are performed utilizing an off-the-shelf PC and the like. The modules also may be implemented as software modules within a processing unit. Furthermore, the diagnostic module 136 may include the therapy module 125 (FIG. 1).

The operations of the modules illustrated in FIGS. 2 and 3 may be controlled by a local ultrasound controller 150 or by the diagnostic module 136. The modules 152-166 perform mid-processor operations. The diagnostic module 136 may receive ultrasound data 170 in one of several forms. In the embodiment of FIG. 2, the received ultrasound data 170 constitutes IQ data pairs representing the real and imaginary components associated with each data sample. The IQ data pairs are provided to one or more modules, for example, a color-flow module 152, an acoustic radiation force imaging (ARFI) module 154, a B-mode module 156, a spectral Doppler module 158, an acoustic streaming module 160, a tissue Doppler module 162, a C-scan module 164, and an elastography module 166. Other modules may be included, such as an M-mode module, power Doppler module, harmonic tissue strain imaging, among others. However, embodiments described herein are not limited to processing IQ data pairs. For example, processing may be done with RF data and/or using other methods. Furthermore, data may be processed through multiple modules.

Each of the modules 152-166 are configured to process the IQ data pairs in a corresponding manner to generate color-flow data 172, ARF1 data 174, B-mode data 176, spectral Doppler data 178, acoustic streaming data 180, tissue Doppler data 182, C-scan data 184, elastography data 186, among others, all of which may be stored in a memory 190 (or memory 134 or image memory 140 shown in FIG. 1) temporarily before subsequent processing. The data 172-186 may be stored, for example, as sets of vector data values, where each set defines an individual ultrasound image frame. The vector data values are generally organized based on the polar coordinate system.

A scan converter module 192 accesses and obtains from the memory 190 the vector data values associated with an image frame and converts the set of vector data values to Cartesian coordinates to generate an ultrasound image frame 193 formatted for display. The ultrasound image frames 193 generated by the scan converter module 192 may be provided back to the memory 190 for subsequent processing or may be provided to the memory 134 (FIG. 1) or the image memory 140 (FIG. 1). Once the scan converter module 192 generates the ultrasound image frames 193 associated with the data, the image frames may be restored in the memory 190 or communicated over a bus 199 to a database (not shown), the memory 134, the image memory 140 and/or to other processors (not shown).

As an example, it may be desired to view different ultrasound images relating to a therapy session in real-time on the display 138 (FIG. 1). To do so, the scan converter module 192 obtains data sets for images stored in the memory 190 of that are currently being acquired. The vector data is interpolated where necessary and converted into an X,Y format for video display to produce ultrasound image frames. The scan converted ultrasound image frames are provided to a display controller (not shown) that may include a video processor that maps the video to a gray-scale mapping for video display. The gray-scale map may represent a transfer function of the raw image data to displayed gray levels. Once the video data is mapped to the gray-scale values, the display controller controls the display 38, which may include one or more monitors or windows of the display, to display the image frame. The image displayed in the display 138 is produced from an image frame of data in which each datum indicates the intensity or brightness of a respective pixel in the display.

Referring again to FIG. 2, a 2D video processor module 194 may be used to combine one or more of the frames generated from the different types of ultrasound information. For example, the 2D video processor module 194 may combine different image frames by mapping one type of data to a gray map and mapping the other type of data to a color map for video display. In the final displayed image, the color pixel data is superimposed on the gray scale pixel data to form a single multi-mode image frame that is again re-stored in the memory 190 or communicated over the bus 199. Successive frames of images may be stored as a cine loop (41) images) in the memory 190 or memory 140 (FIG. 1). The cine loop represents a first in, first out circular image buffer to capture image data that is displayed in real-time to the user. The user may freeze the cine loop by entering a freeze command at the user interface 142. The user interface 142 may include, for example, a keyboard and mouse and all other input controls associated with inputting information into the ultrasound system 120 (FIG. 1). In one embodiment, the user interface 142 includes the display 138 that may be touch-sensitive or configured to interact with a stylus.

A 3D processor module 196 is also controlled by the user interface 142 and accesses the memory 190 to obtain spatially consecutive groups of ultrasound image frames and to generate three dimensional image representations thereof, such as through volume rendering or surface rendering algorithms as are known. The three dimensional images may be generated utilizing various imaging techniques, such as ray-casting, maximum intensity pixel projection and the like.

A graphic module 197 is also controlled by the user interface 142 and accesses the memory 190 to obtain groups of ultrasound image frames that have been stored or that are currently being acquired. The graphic module 197 may generate images that include the images of the ROI and a graphical representation positioned (e.g., overlaid) onto the images of the ROI. The graphical representation may represent an outline of a treatment space, the focal region of the therapy beam, a path taken by the focal region within the treatment space, a probe used during the session, and the like. Graphical representations may also be used to indicate the progress of the therapy session. The graphical representations may be generated using a saved graphical image or drawing (e.g., computer graphic generated drawing), or the graphical representation may be directly drawn by the user onto the image using a pointing device, e.g., an electronic stylus or mouse, or another interface device.

Also shown, a reference module 195 may be used to identify a reference point on the patient during the therapy session. For example, a reference point may be an anatomical element or structure of the body that is determined by the system 120 or by the user. The reference point may also be an element or marker positioned on the surface of the body of the patient. As will be described in greater detail below, the reference module 195 may use the imaging data to determine a relation of the treatment space with respect to a reference point.

FIG. 4 illustrates a window 202 that may be presented on the display 138 (FIG. 1). The display 138 communicates with the diagnostic module 136 (FIG. 1) to display an image 204 of the ROI of the patient within the window 202. As shown in the image 204, the ROI may include adipose layers or tissues 206 and 208 and non-adipose layers or tissues 209 (e.g., dermis layer) and 210 (e.g., muscle tissue). The user of the system 120 may be able to recognize through the image 204 a boundary between the layers. Also, the system 120 may be able to automatically identify or differentiate between the layers as described in the patent Application having Attorney Docket No. 235615 (555-0004US), which is incorporated by reference in the entirety. In some embodiments, the user interface 142 (FIG. 1) accepts user inputs for designating a treatment space 212 within the ROI. The treatment space 212 represents an area or region that will be treated during a therapy session and is generally located within the adipose tissue 206.

A “therapy session,” as used herein, is a period of time in which a patient receives therapy. For example, a therapy session may include a single application of ultrasounds signals to liquefy adipose tissue at a single treatment location or within a single treatment space within the body. A therapy session may also include an extended period of time in which a patient receives multiple applications of ultrasound signals within a treatment space of one region of the body or within multiple regions of the body. A therapy session may also include one visit by a patient to an operator of the system 120.

The diagnostic module 136 may be configured to acquire the diagnostic ultrasound signals at different frame rates. A frame rate is the number of frames or images taken per second. More specifically, the diagnostic module 136 may be configured to acquire diagnostic ultrasound signals associated with different imaging areas within the ROI at different frame rates. For example, signals from the treatment space 212 may be acquired at one frame rate while signals from other areas or regions outside of the treatment space 212 may be acquired at another frame rate. In one embodiment, the diagnostic module 136 is configured to acquire diagnostic ultrasound signals at a first rate in an imaging area that includes the treatment space 212 and at a slower second rate in an imaging area that excludes the treatment space 212. Alternatively, the first rate may be slower than the second rate.

The treatment space 212 may correspond to a portion of the adipose tissue 206 within the image 204 or the treatment space 212 may correspond to all of the adipose tissue 206 within the ROI. By way of example, the treatment space 212 may be located and shaped so that the treatment space 212 is a distance away from the non-adipose tissue 209 and 210. As such, the system 120 (FIG. 1) may decrease the probability of therapy being inadvertently applied to areas outside of the treatment space 212, such as the non-adipose tissue 210.

The display 138 may indicate to the user or another viewer the treatment space 212 designated by the user inputs. A graphical representation, such as an outline 214, may be overlaid upon the image 204. The outline 214 designates boundaries of the treatment space 212 to indicate to a viewer where the therapy will be applied. The outline 214 may be determined by parameters entered by the user. For example, the user may select pre-programmed outlines 214 or may enter coordinates or dimensions for the treatment space 212 to form the outline 214. The outline 214 may indicate an enclosed region within the treatment space 212. The outline 214 may have various shapes including a rounded rectangular shape (as shown), a parallelogram shape, another geometric shape, and the like, or a shape determined by the system 120.

The user may also enter a drawing notation to indicate where the outline 214 should be located. The drawing notation may be entered through a keyboard, a mouse, or another pointing device. As an example, the user may use a stylus pen and directly contact a touch-sensitive screen of the display 138 or a pad that is communicatively coupled to the user interface 142 to draw the drawing notation onto the image 204. As another example, the user interface 142 may recognize touches from a finger to the screen of the display 138. Furthermore, the user interface 142 may have a voice-activation module that receives voice commands from the user for entering user inputs including the drawing notation.

The reference module 195 (FIG. 2) may be configured to identify a reference point 250, 252, or 254 on the patient or receive user inputs that identify the corresponding reference point. For instance, the reference point 250 may be a surface of the patient's skin, the reference point 252 may be a particular point of or a portion of a boundary between the adipose tissues 206 and 208, and the reference point 254 may be a point along a surface of the probe 126. Reference points may also be other points within the ROI, such as bone, other artifacts, or a reference element such as a metallic sticker placed on a patient's skin.

After identifying a reference point, the reference module 195 may determine a relation of the treatment space 212 with respect to the reference point using ultrasound signal processing methods (e.g., speckle tracking). The reference module 195 may position the outline 214 of the treatment space 212 on the image 204 based on the relation of the treatment space 212 with respect to the reference point. As a more specific example, the reference module 195 may establish a positional relation between the adipose tissue 206 and the reference point 254 that represents a surface of the probe 126. Based on the positional relation, the reference module 195 may adjust a position of the treatment space 212 on the image 204. In other words, as the probe 126 moves along the surface of the skin or is pressed into the patient, the outline 214 on the image 204 may also move.

In some embodiments, the system 120 may automatically differentiate the adipose tissues 206 and 208 and the non-adipose tissue 210. The system 1120 may also automatically display to a viewer a boundary between the adipose tissue 206 and 208 and between the adipose tissue 206 and the non-adipose tissue 210 by overlaying the image 204 with a graphical representation that indicates the boundary. Furthermore, the system 120 may automatically display to a viewer of the system 120 the treatment space 212 within the image 204 where therapy may be applied (or is recommended by the system 120 to be applied). In addition, the user may be able to modify the treatment space 120 that was automatically displayed by the system 120 through user inputs. Such automatic functions are described in greater detail in the U.S. patent Application having Attorney Docket No. 235615 (555-0004), filed contemporaneously herewith, which is incorporated by reference in the entirety.

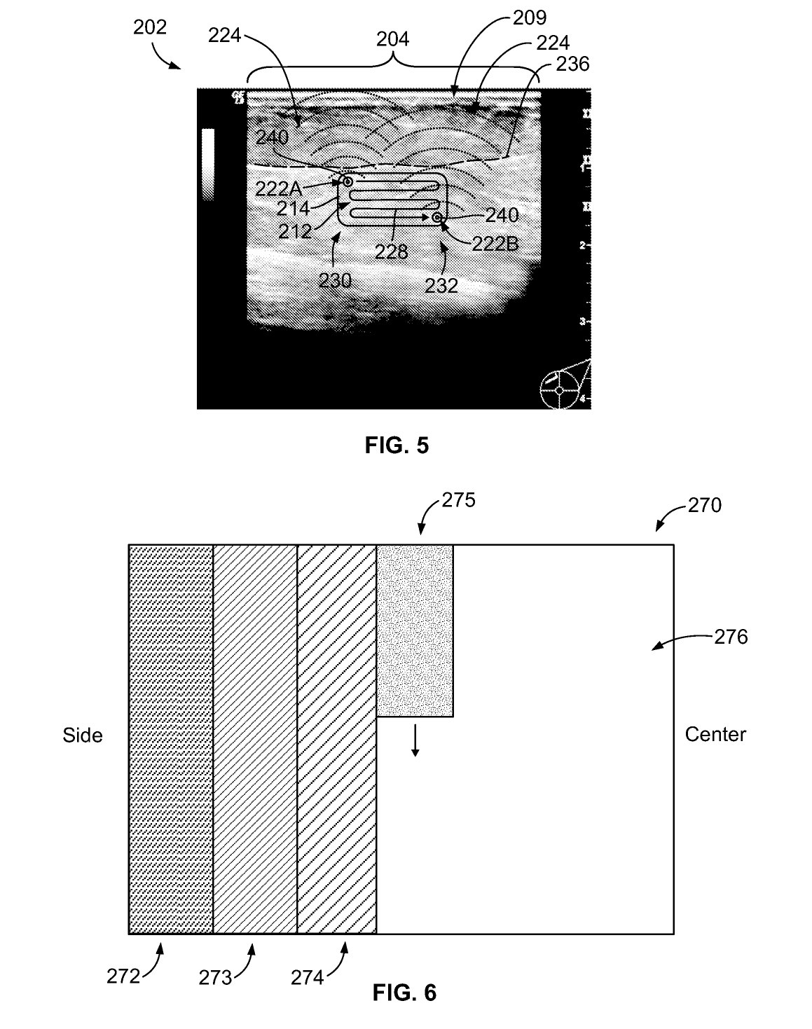

FIG. 5 shows the window 202 as the system 120 (FIG. 1) delivers therapy to the treatment space 212. When therapy is applied, ultrasonic therapy signals (e.g. HIFU) from the probe 126 (FIG. 1) are directed toward a treatment location 222 (indicated as dots 222A and 222B in FIG. 5) within the treatment space 212. A treatment location 222 includes a region where a therapy beam 224 formed by ultrasound signals from the transducer elements 124 is focused (i.e., the treatment location 222 includes a focal region of the transducer elements, 124) within a body of a patient. The therapy beam 224 is shaped and directed by a selected configuration and operation of the transducer elements 124. As such, the treatment location 222 may vary in size and shape within a single therapy session. When the adipose tissue 206 is treated, the therapy beam 224 that is delivered to the treatment location 222 at least partially liquefies (e.g., lyses, causes cavitation and/or thermal damage) the adipose tissue 206 within the focal region. Adipose tissue within a space that immediately surrounds the focal region may also be affected.

The therapy module 125 (FIG. 1) is configured to move the treatment location 222 throughout the treatment space 212 between multiple points or treatment sites. As used herein, “moving the treatment location between multiple points” includes moving the treatment location 222 along a therapy path 228 between a first point and an end point and also includes moving the treatment location 222 to separate and distinct points within the treatment space 212 that may or may not be adjacent to one another along a path. The therapy path 228 may be formed by separate points where therapy is applied. For example, therapy may first be applied to a first point (indicated as the treatment location 222A). After therapy has been applied to the first point, the focal region may be readjusted onto a second point along the therapy path 228 that is separate and remotely spaced from the first point. Therapy may then be applied to the second point. The process may continue along the therapy path 228 until the therapy session is concluded at an end point (indicated as the treatment location 222B). In other embodiments, the therapy may be continuously applied as the focal region is moved along the therapy path 228 in a sweeping manner. For example, therapy may be continuously applied as the treatment location 222 is moved between the first point and the end point in FIG. 5.

The therapy path 228 may have various shapes and may be pre-programmed or, alternatively, drawn by the user. As shown in FIG. 5, the therapy module 125 may direct the treatment location 222 in a sweeping manner within the treatment space 212. More specifically, the treatment location 222 may move from a first lateral location 230 proximate one side of the image 204 or outline 214 to a second lateral location that 232 is proximate an opposing side of the image 204 or the outline 214. The treatment location 222 may maintain a predetermined depth within the adipose tissue 206 as the treatment location 222 moves between the first and second lateral locations 230 and 232. In some embodiments, after the treatment location 222 is moved from the first lateral location 230 to the second lateral location 232, the depth of the treatment location 222 may be increased or decreased. As shown in FIG. 5, the treatment location 222 moves back and forth between the first and second lateral locations 230 and 232 and increases a depth of the treatment location 222 after each crossing of the treatment space 212. As such, portions of the adipose tissue 206 may avoid sustaining multiple periods of therapy. Alternatively, the depth of the treatment location 222 may gradually change as the treatment location 222 is moved in a sweeping manner. As an example, the depth of the treatment location 222 within the adipose tissue 206 may move parallel to a boundary 236 (indicated as a dashed line) between the adipose tissues 206 and 208. The boundary 236 may or may not be shown to the viewer.

However, the therapy path 228 shown in FIG. 5 is just one example of applying therapy to multiple points within the treatment space 212. Many other therapy paths may be taken by the treatment location 222. For example, the therapy module 125 may direct the treatment location 222 in a sweeping manner between two vertical locations while changing a lateral position within the treatment space 212 after the vertical locations have been traversed. Furthermore, the treatment location 222 is not required to move between adjacent points along the therapy path 228, but may be moved to predetermined or random points within the treatment space 212 that are not adjacent to each other. For example, therapy may be applied to one corner of a treatment space 212. Subsequently, the focal region may then be readjusted to another corner and therapy may be applied.

In some embodiments, the therapy path 228 is at least partially determined by a therapy parameter. For example, a shape of the focal region or a thickness of the adipose tissue to be treated may determine the therapy path taken.

However, in alternative embodiments, the treatment location 222 may be manually moved or steered along a therapy path by the user of the system 120. The user may view the display 138 while applying therapy within the treatment space 212 and the display 138 may indicate to the user where the treatment location 222 is located. For example, as will be described in greater detail below, the display 138 may show a marker 240 that indicates where the treatment location 222 is presently located within the treatment space 212. Furthermore, in some embodiments, the marker 240 may move within the treatment space 212 independently or, alternatively, the outline 214 may move with the marker 240 such that the marker 240 is always located at a predetermined location within the outline 214.

Returning to FIG. 5, in some embodiments, the display 138 may overlay another graphical representation, such as a marker 240, onto the image 204 that designates the treatment location or locations 222. The size and shape of the marker 240 may correspond to a size and shape of the focal region of the probe 126. As the therapy beam 224 moves the treatment location 222 within the treatment space 212, the display 138 may continuously update the marker 240 to cover new points within the treatment space 212 as the new points are receiving the therapy. In some embodiments, the marker 240 may only correspond to the point or points within the treatment space that are currently receiving treatment.

However, in other embodiments, the marker 240 or another graphical representation may also indicate a path within the treatment space 212 that has received therapy. For example, if the treatment location 222 is applied continuously and moved within the treatment space 212, the path may be indicated by a thick line (e.g. like a paint stroke) along the path. If the therapy is applied at separate and distinct points, a graphical representation, such as the marker 240, may be left on each point. As such, at an end of the therapy session, the image 204 may have multiple markers 240 overlaid upon the image 204 that indicate where therapy has been applied. In some embodiments, the graphical representations that indicate past therapy may remain on the image 204 indefinitely (i.e., until removed by the user or until the therapy session has concluded). In other embodiments, the graphical representations indicating past therapy may change as time progresses. Such graphical representations may indicate a time since therapy was applied, a fluidity of the tissue, a temperature, tissue stiffness, or some other characteristic of the tissue that may change with time. As an example, when therapy is first applied to a point, the graphical representation may be red to indicate that the point has recently received therapy. As time progresses, the graphical representation may fade or change into another color (e.g., blue) to indicate a predetermined amount of time has passed since therapy was applied to the point.

FIG. 6 is an image 270 of a C-plane view of the ROI at a predetermined depth. A C-plane view extends along a plane that does not intersect the probe 126 or the transducer elements 124. The C-plane view may be perpendicular to the view of the image 204 shown in FIGS. 4 and 5. In some embodiments, the C-plane view of the ROI is used in conjunction with the image 204. The image 270 may be provided in a window (not shown) on the display 138 concurrently with the window 202 or separately. The image 270 may also be presented on a separate display (not shown). In alternative embodiments, the image 270 is used exclusively during a therapy session.

The C-plane view in FIG. 6 shows an ultrasound image along the C-plane at a predetermined depth. The following is with respect to one depth with the ROI. However, after therapy has been applied to one depth of the ROI, the user of the system 120 (FIG. 1) may change depths and obtain a new C-plane view at the new depth. As shown, the image 270 illustrates sections 272-275 that indicate those areas or regions within the view of the image 270 that have completed treatment or a portion of treatment. Section 276 has not received any treatment. More specifically, the image 270 may show a patient's abdomen region. Section 272 is proximate to a side of the patient and section 275 is proximate to a center (e.g., navel) of the patient. During a therapy session, a user may apply therapy to section 272 near the patient's side. As similarly described above with respect to the marker 240, the image 270 may indicate to the user those areas of the abdomen region that have already completed treatment. Furthermore, through ultrasound signal processing methods, the sections 272-275 may have different characteristics, such as different or contrasting colors. Section 275 may have a characteristic that indicates therapy is being currently provided or was recently provided. The section 272 may have a characteristic that indicates therapy was applied therein a period of time ago.

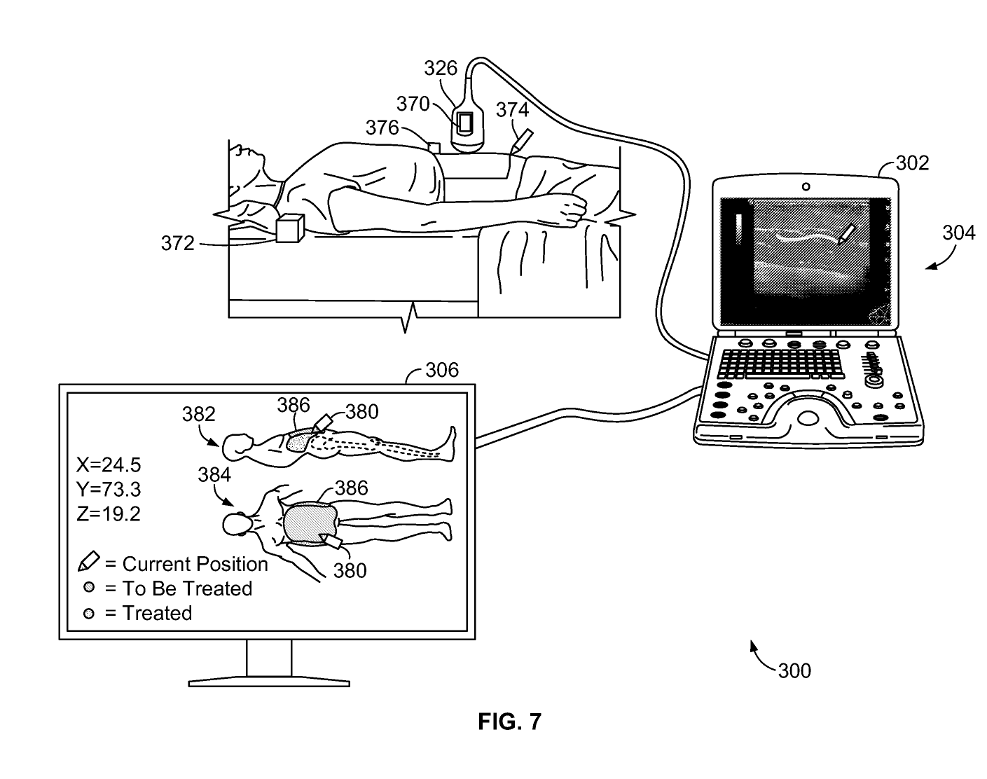

FIG. 7 illustrates an ultrasound system 300 formed in accordance with one embodiment. The system 300 may include similar features and components as described above with respect to FIGS. 1-6. More specifically, the system 300 includes a portable computer 302 that has a primary display 304 and that is communicatively coupled to a secondary display 306. The computer 302 may also include software and internal circuitry configured to perform as described above with respect to the system 120 (FIG. 1). The system 300 includes a probe 326 that is coupled to the computer 302 and has a probe position device 370. The system also includes a reference position device 372 that may be located near the patient or may be attached to the patient. The position devices 370 and 372 may have transmitters and/or receivers that communicate with each other and/or with the computer 302. For example, the position devices 370 and 372 may communicate with a position tracking module (not shown), such as the position tracking module 148 shown in FIG. 1. The position tracking module may receive signals from the position devices 370 and/or 372. In one particular embodiment, the position device 372 has a pair of coils that creates an electromagnetic field. The position tracking module receives data (e.g., positional information) from the position devices 370 and 372 regarding a location of the probe 326. As the probe 326 applies therapy to the patient and is moved along the patient, the display 304 and/or 306 may show the movement of the probe 326 with respect to the patient.

Also shown in FIG. 7, the system 300 may be configured to register where therapy will be applied during the therapy session. The system 300 may include an electronic pen 374 and fiducial element 376 attached to the body of the patient. The fiducial element 376 is attached near the sternum of the patient in FIG. 7, but may be attached to other areas. A user desiring to outline or delineate where therapy will be applied may use the electronic pen 374 to draw on the body of the patient. First, the electronic pen 374 may register with the fiducial element 376 so that the location of the electronic pen 374 with respect to the body of the patient is known. After registering, the electronic pen 374 moves along the surface of the body and communicates with the computer 302 a current position of the electronic pen 374. Also, the electronic pen 374 may mark the patient's body (e.g., through ink, resin, or another substance) where therapy will be applied. The computer 302 uses the data received by the electronic pen 374 and the position device 372 to indicate on the display 306 where therapy is to be applied. As shown, the display 306 may show a graphical representation 382 of a side-view of the body and a graphical representation 384 of an anterior view of the body. The computer 302 uses the information from the electronic pen 374 to outline a region 386 of the body to be treated. The region 386 may be colored green prior to treatment. In an alternative embodiment, a single element or device may perform the functions of the fiducial element 376 and the reference position device 372.

As one example, the graphical representations 382 and 384 may be digital photographs of the patient's body. When therapy is applied to the body, the computer 302 tracks the position of the probe 326. As therapy is applied, the display 306 indicates an overall progress of the therapy session. For example, the display 306 may show the user the region of the body that is currently receiving therapy, the regions of the body that have already received therapy, and the regions of the body that have yet to receive therapy. For example, the regions that have received therapy may be colored red and the regions that have not received therapy may be colored green. Also, a graphical representation 380 of the probe 326 may be shown on the display 306 to indicate a current position of the probe 326 with respect to the body.

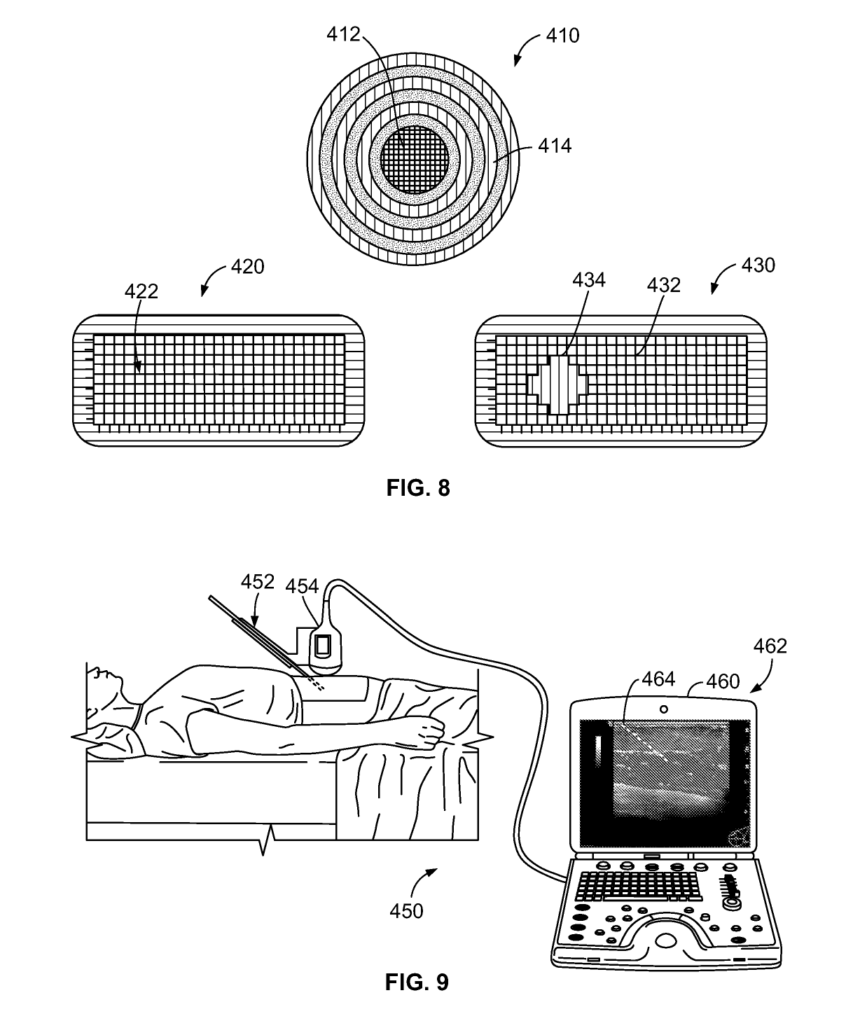

FIG. 8 illustrates transducers 410, 420, and 430 that may be used with a probe (not shown) in accordance with various embodiments. The transducers 410, 420, and 430 may include reconfigurable arrays. In some embodiments, the diagnostic module 136 (FIG. 1) and the therapy module 125 (FIG. 1) control the probe 126 (FIG. 1) to deliver low energy imaging pulses and high energy therapy pulses, respectively. More specifically, the transducer 410 has an imaging array 412 and a separate therapy array 414 that surrounds the imaging array 412. The imaging pulses and the therapy pulses may be delivered separately or in an overlapping manner. The transducer 420 includes an array 422 where the entire array may be used for both imaging and therapy. However, the transducer 430 has an array 432 of transducer elements where a therapy portion 434 of the array 432 may be activated to provide therapy. As such, the therapy module 125 may drive a subset (e.g., the therapy portion 434) of the transducer elements of the array 432 based on the user inputs designating the treatment space. Thus, the diagnostic module 136 and the therapy module 125 may deliver low energy imaging pulses and high energy therapy pulses in an interspersed manner to an at least partially overlapping array of transducer elements.

When imaging or applying therapy to a patient, the pressure applied by the transducer to the patient's body may alter the thickness or other characteristics of the ROI, such as tissue stiffness. By combining the imaging and therapy arrays into one transducer, therapy may be applied immediately after the transducer images the ROI. As such, an accurate representation or identification of the adipose tissue may be provided immediately before the therapy is applied.

FIG. 9 illustrates an ultrasound system 450 in accordance with one embodiment that includes a device 452 for removing tissue or liquid from a patient during a therapy session. The device 452 may include a hollow tube that is inserted into the body of the patient (i.e., beneath the skin of the patient where proximate to where therapy is being received). The device 452 may also include a suction device (not shown) for removing the tissue or liquid from within the ROI through the tube. The probe 454 is communicatively coupled to a computer 460 having a display 462. The display 462 may show the tube or provide a graphical representation 464 of the tube during a therapy session.

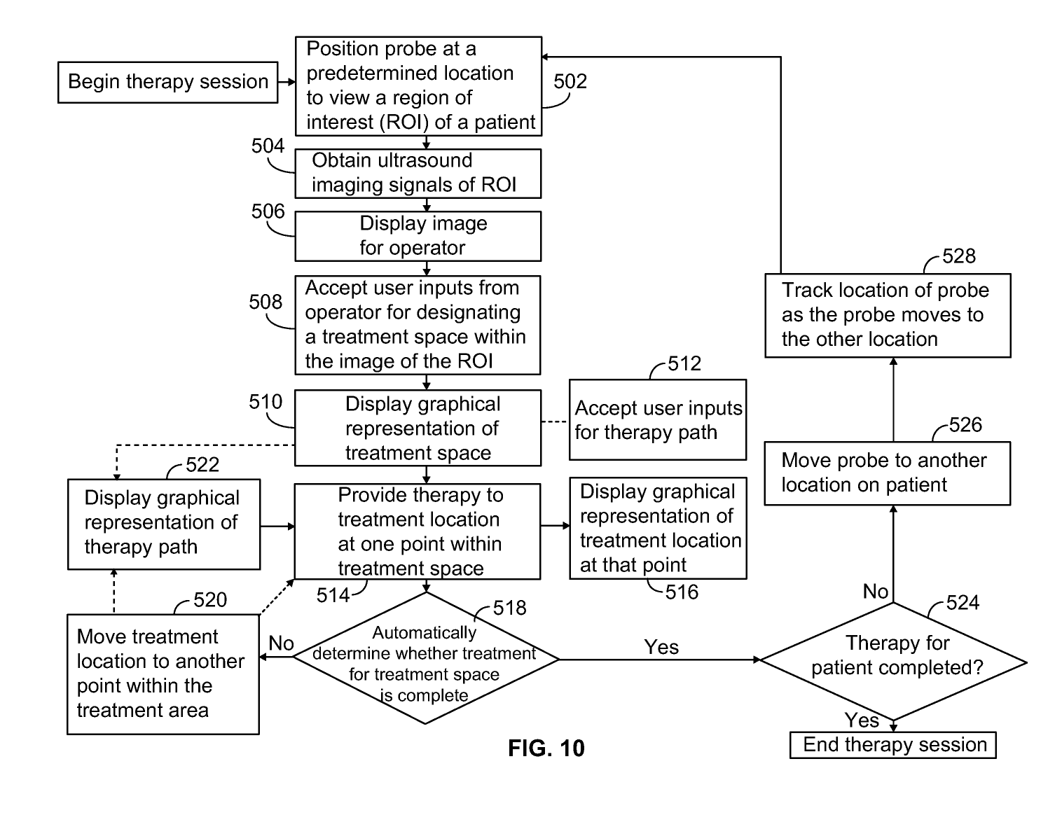

FIG. 10 is a flowchart illustrating a method 500 for delivering therapy to at least one ROI in a patient. The method 500 may be performed by a user or an operator of an imaging and therapy system. For example, the system used may be the systems 120, 300, or 450 (discussed above) or other systems described below. The therapy session may begin when, at step 502, the operator positions a probe at a predetermined location on the body of the patient to view an ROI. The ROI may be one of many that will be viewed during the therapy session. At step 504, ultrasound imaging signals of the ROI are obtained. The signals may be processed into data via different ultrasound sub-modules, such as the modules 152-166 described above with reference to FIG. 2. In one embodiment, the signals are processed into data via elastography methods.

At step 506, an image of the ROI is generated and displayed to the operator and, optionally, patient. When the image is displayed, the system may automatically identify and indicate to the operator the different layers of tissue within the image. For example, the system may automatically overlay a graphical presentation (e.g., line) that indicates a boundary between the layers of tissue. Alternatively, the system simply shows the ultrasound image without any graphical presentations. The operator may enter user inputs via a user interface into the system. At step 508, the system may accept the user inputs from the operator that designate a treatment space within the image of the ROI. In some embodiments, once the treatment space is indicated, the system may process the signals obtained from the treatment space via different processing methods than the area not within the designated treatment space.

At step 510, the system may display a graphical representation (e.g., an outline of a rectangle or some other geometric shape) of the designated treatment space. The operator may then enter user inputs, such as therapy parameters, before providing therapy. Optionally, at step 512, the operator may designate a therapy path within the treatment space. Then, at step 514, therapy is provided to a treatment location within the designated treatment space. In the illustrated embodiment, the treatment is provided to one point within the treatment space. The system may optionally, at step 516, display a graphical representation (e.g., a marker) of the treatment location with the image.

After or while providing treatment to the one point within the treatment space, the system may automatically determine, at step 518, whether treatment is complete for the treatment space and if the treatment location should be moved to another point within the treatment space. Automatic determination of whether the treatment space has been sufficiently treated or completed may be determined by, for example, elastographic methods. Alternatively, the user of the system may determine that treatment is complete. If treatment for the corresponding treatment space is not complete, the system may automatically move, at step 520, the treatment location to another point within the treatment space. The treatment location may move while providing treatment or after treatment has ended for a particular point. Optionally, the system may display a graphical representation that indicates the path taken by the treatment location within the treatment space at step 522. The system then provides therapy to the new point and continues this process until the therapy for the corresponding treatment space is complete.

After therapy for the treatment space is complete, the system may determine (or ask the operator), at step 524, whether therapy for the patient is complete. If therapy for the patient is complete, then the therapy session has ended. However, if the therapy session is not complete, then at step 526 the system or the operator may move the probe to another location on the patient. In some embodiments, the system may also track, at step 528, a location of the probe as the probe moves to another location. Furthermore, the system may also display to the operator those regions that have already received treatment and those regions that have not received treatment.

Although the flowchart illustrates sequential steps in the method 500, embodiments herein include methods that perform fewer steps and also methods that perform the steps in different orders or may perform steps simultaneously. For example, the system may also provide therapy to a treatment location within the ROI and simultaneously obtain imaging signals and display an image of the ROI during the therapy.

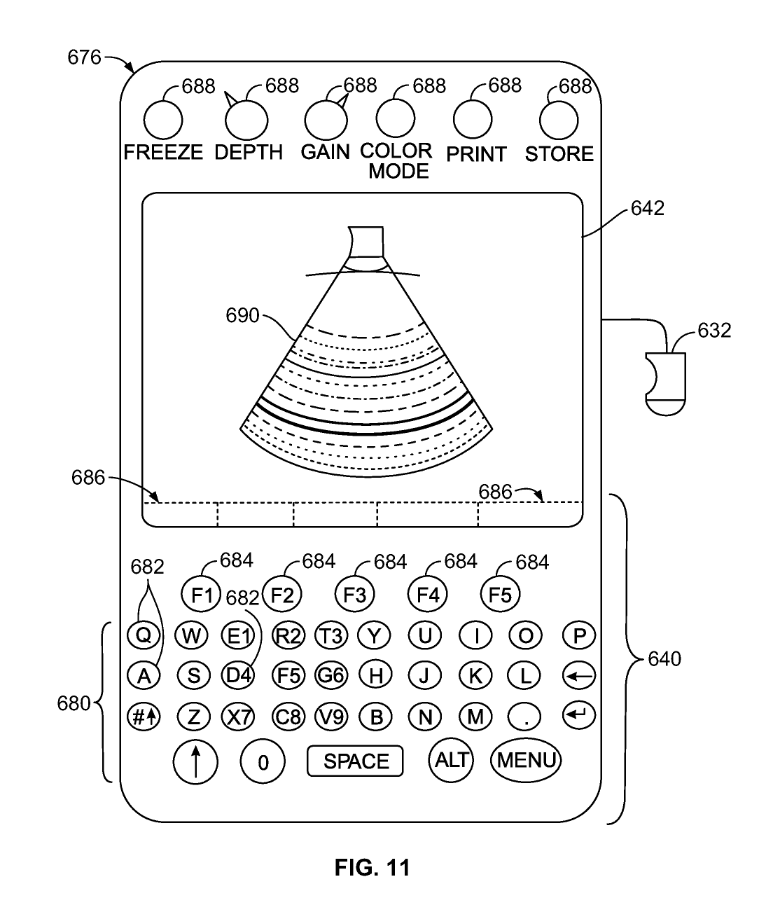

FIG. 11 shows another example of an ultrasound system and, in particular, a hand carried or pocket-sized ultrasound imaging system 676. In the system 676, a display 642 and a user interface 640 form a single unit. By way of example, the pocket-sized ultrasound imaging system 676 may be a pocket-sized or hand-sized ultrasound system approximately 2 inches wide, approximately 4 inches in length, and approximately 0.5 inches in depth and weighs less than 3 ounces. The display 642 may be, for example, a 320×320 pixel color LCD display (on which a medical image 690 may be displayed in combination with a graphical representation(s) as described above). A typewriter-like keyboard 680 of buttons 682 may optionally be included in the user interface 640. It should be noted that the various embodiments may be implemented in connection with a pocket-sized ultrasound system 676 having different dimensions, weights, and power consumption.

Multi-function controls 684 may each be assigned functions in accordance with the mode of system operation. Therefore, each of the multi-function controls 684 may be configured to provide a plurality of different actions. Label display areas 686 associated with the multi-function controls 684 may be included as necessary on the display 642. The system 676 may also have additional keys and/or controls 688 for special purpose functions, which may include, but are not limited to “freeze,” “depth control,” “gain control,” “color-mode,” “print,” and “store.”

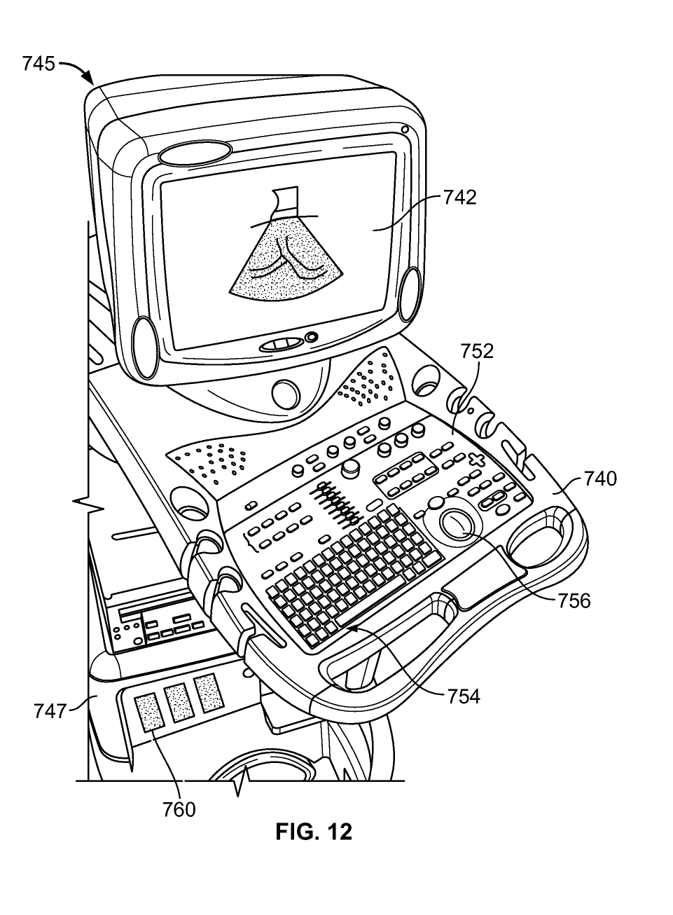

As another example shown in FIG. 12, a console-based ultrasound system 745 may be provided on a movable base 747 that may be configured to display the region of interest during a therapy session. The system 745 may also be referred to as a cart-based system. A display 742 and user interface 740 are provided and it should be understood that the display 742 may be separate or separable from the user interface 740. The user interface 740 may optionally be a touchscreen, allowing the operator to select options by touching displayed graphics, icons, and the like.

The user interface 740 also includes control buttons 752 that may be used to control the portable ultrasound imaging system 745 as desired or needed, and/or as typically provided. The user interface 740 provides multiple interface options that the user may physically manipulate to interact with ultrasound data and other data that may be displayed, as well as to enter user inputs and set and change imaging or therapy parameters. The interface options may be used for specific inputs, programmable inputs, contextual inputs, and the like. For example, a keyboard 754 and track ball 756 may be provided. The system 745 has at least one probe port 760 for accepting probes.

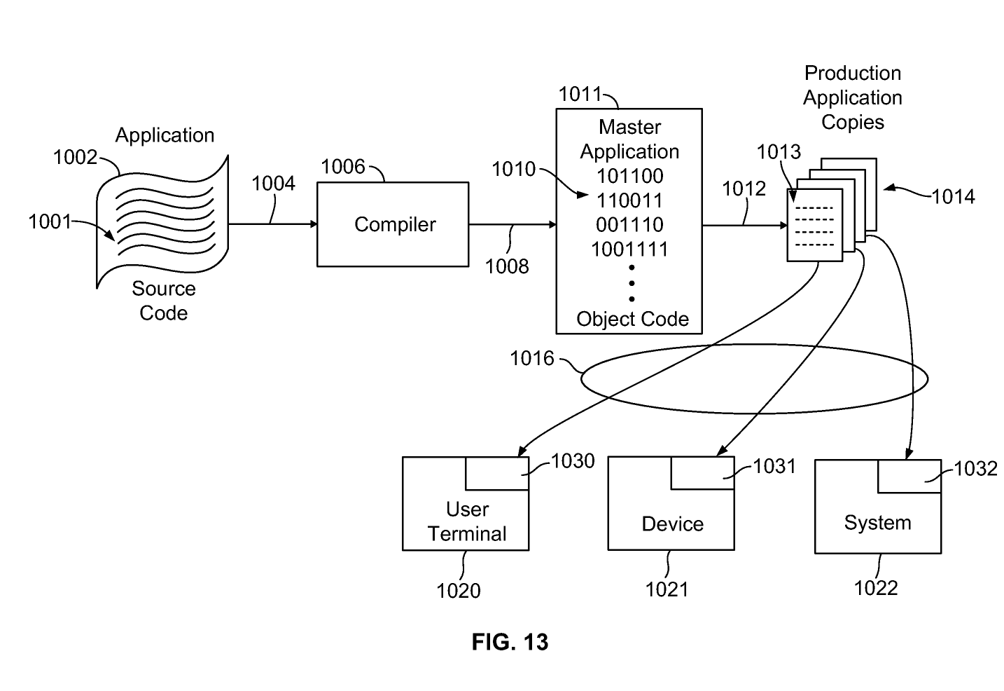

FIG. 13 is a block diagram of exemplary manners in which various embodiments described herein may be stored, distributed and installed on computer readable medium. In FIG. 13, the “application” represents one or more of the methods and process operations discussed above.

As shown in FIG. 13, the application is initially generated and stored as source code 1001 on a source computer readable medium 1002. The source code 1001 is then conveyed over path 1004 and processed by a compiler 1006 to produce object code 1010. The object code 1010 is conveyed over path 1008 and saved as one or more application masters on a master computer readable medium 1011. The object code 1010 is then copied numerous times, as denoted by path 1012, to produce production application copies 1013 that are saved on separate production computer readable medium 1014. The production computer readable medium 1014 is then conveyed, as denoted by path 1016, to various systems, devices, terminals and the like. In the example of FIG. 13, a user terminal 1020, a device 1021 and a system 1022 are shown as examples of hardware components, on which the production computer readable medium 1014 are installed as applications (as denoted by 1030-1032).

The source code may be written as scripts, or in any high-level or low-level language. Examples of the source, master, and production computer readable medium 1002, 1011 and 1014 include, but are not limited to, CDROM. RAM, ROM, Flash memory, RAID drives, memory on a computer system and the like. Examples of the paths 1004, 1008, 1012, and 1016 include, but are not limited to, network paths, the internet, Bluetooth, GSM, infrared wireless LANs, HIPERLAN, 3G, satellite, and the like. The paths 1004, 1008, 1012, and 1016 may also represent public or private carrier services that transport one or more physical copies of the source, master, or production computer readable medium 1002, 1011, or 1014 between two geographic locations. The paths 1004, 1008, 1012, and 1016 may represent threads carried out by one or more processors in parallel. For example, one computer may hold the source code 1001, compiler 1006 and object code 1010. Multiple computers may operate in parallel to produce the production application copies 1013. The paths 1004, 1008, 1012, and 1016 may be intra-state, inter-state, intra-country, inter-country, intra-continental, inter-continental and the like.

As used throughout the specification and claims, the phrases “computer readable medium” and “instructions configured to” shall refer to any one or all of i) the source computer readable medium 1002 and source code 1001, ii) the master computer readable medium and object code 1010, iii) the production computer readable medium 1014 and production application copies 1013 and/or iv) the applications 1030-1032 saved in memory in the terminal 1020, device 1021 and system 1022.

The various embodiments and/or components, for example, the monitor or display, or components and controllers therein, also may be implemented as part of one or more computers or processors. The computer or processor may include a computing device, an input device, a display unit, and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes described herein. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

Although the embodiments described above are illustrated as treating adipose tissue, alternative embodiments may be used to treat other tissues within the body. For example, the above described embodiments may be used to image and treat a tumor within a region of interest. As described above with respect to adipose tissue, embodiments may be used to automatically identify the tumor and/or to allow user inputs to identify treatment spaces within a region of interest and to set therapy parameters for the treatment. Furthermore, embodiments described herein may be used for palliative treatments for cancer, thermal treatment of muscles, or ultrasonically activating drugs, proteins, stem cells, vaccines. DNA, and gene delivery.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasound imaging and therapy system, comprising:
an ultrasound probe;
a diagnostic module to control the probe to obtain diagnostic ultrasound signals from a region of interest (ROI) of the patient, the ROI including adipose tissue, the diagnostic module generating a diagnostic image of the ROI based on the ultrasound signals obtained;
a display to display the image of the ROI;
a user interface to accept user inputs to designate a treatment space within the ROI that corresponds to the adipose tissue, the display displaying the treatment space on the image; and
a therapy module to control the probe to deliver, during a therapy session, a therapy to a treatment location based on a therapy parameter, the treatment location being within the treatment space defined by the user inputs.

2. The system in accordance with claim 1 wherein the therapy module is configured to automatically move the treatment location between multiple points within the treatment space.

3. The system in accordance with claim 1 wherein the display displays an outline overlaid upon the image of the ROI, the outline designating boundaries of the treatment space defined by the user inputs.

4. The system in accordance with claim 1 wherein the display displays a marker overlaid upon the image, the marker designating the treatment locations that have received the therapy, the display continuously updating the marker to cover new treatment sites of the treatment space as the therapy is applied to the treatment sites.

5. The system in accordance with claim 1 further comprising a reference module to identify a reference point on the patient, the reference module determining a relation of the treatment space with respect to the reference point, the reference module positioning the outline of the treatment space on the image based on the relation of the treatment space with respect to the reference point.

6. The system in accordance with claim 1 wherein the therapy module directs the probe to generate a therapy beam and to sweep a treatment location of the therapy beam across the treatment space.

7. The system in accordance with claim 1 wherein the image displayed represents a C-plane view of the ROI, the C-plane view extending along a plane that does not intersect the probe.

8. The system in accordance with claim 1 further comprising a reference module to establish a positional relation between the adipose tissue and a surface of the probe, based on the positional relation, the reference module adjusting a position of the treatment space on the image.

9. The system in accordance with claim 1 wherein the user inputs accepted by the user interface includes a drawing notation entered by the user to identify the treatment space.

10. The system in accordance with claim 1 wherein the user interface includes an electronic pen that the user draws on the display with to identify the treatment space.

11. The system in accordance with claim 1 wherein the diagnostic and therapy modules deliver low energy imaging pulses and high energy therapy pulses in an interspersed manner to an at least partially overlapping array of transducer elements.

12. The system in accordance with claim 1 wherein the diagnostic module acquires the diagnostic ultrasound signals at a first rate in an imaging area that includes the treatment space and at a slower second rate in an imaging area that excludes the treatment space.

13. The system in accordance with claim 1 wherein the therapy module drives a subset of transducer elements within an array in the probe, the subset being selected based on the user inputs designating the treatment space.

14. The system in accordance with claim 1 further comprising a position tracking module to track and record movement of the probe with respect to a reference point, the display displaying an overall progress of a therapy including a current probe position, areas to be treated and areas already treated.

15. A method for delivering therapy to a region of interest (ROI) in a patient, the method comprising:
obtaining diagnostic ultrasound signals from the ROI, the ROI including adipose tissue, the diagnostic module generating a diagnostic image of the ROI based on the ultrasound signals obtained;
accepting user inputs to designate a treatment space within the ROI that corresponds to the adipose tissue;
displaying the image and the treatment space on the image on a display, and
providing therapy to a treatment location based on a therapy parameter, the treatment location being within the treatment space defined by the user inputs.

16. The method of claim 15 wherein the step of displaying includes displaying an outline overlaid upon the image of the ROI, the outline designating boundaries of the treatment space.

17. The method of claim 15 wherein the step of displaying includes displaying a marker overlaid upon the image of the ROI, the marker designating the treatment locations that have received the therapy, and continuously updating the marker on the display to cover new treatment locations of the treatment space as the therapy is applied to the treatment locations.

18. The method of claim 15 further comprising identifying a reference point on the patient, determining a relation of the treatment space with respect to the reference point, and positioning an outline of the treatment space on the image based on the relation of the treatment space with respect to the reference point.

19. The method in accordance with claim 15 wherein the providing the therapy includes automatically moving the treatment location between multiple points within the treatment space.

20. The method of claim 15 wherein the image displayed represents a C-plane view of the ROI, the C-plane view extending along a plane that does not intersect the probe.

21. The method of claim 15 further comprising establishing a positional relation between the adipose tissue and a surface of the probe and adjusting a position of the treatment space on the image based on the positional relation.

22. The method of claim 15 wherein the user inputs accepted by the user interface includes a drawing notation entered by the user to identify the treatment space.

23. The method of claim 15 wherein the user interface includes an electronic pen that the user draws on the display with to identify the treatment space.

24. The method of claim 15 wherein the probe includes transducer elements, the probe delivering low energy imaging pulses and high energy therapy pulses in an interspersed manner to an at least partially overlapping array of transducer elements.

25. The method of claim 15 wherein said step of obtaining includes obtaining the diagnostic ultrasound signals at a first rate in an imaging area that includes the treatment space and at a slower second rate in an imaging area that excludes the treatment space.

26. The method of claim 15 wherein said step of providing therapy includes driving a subset of transducer elements within an array in the probe, the subset being selected based on the user inputs designating the treatment space.

27. The method of claim 15 further comprising tracking and recording movement of the probe with respect to a reference point, the display displaying an overall progress of a therapy including a current probe position, areas to be treated and areas already treated.
